(19) United States
(12) Patent Application Publication
(10) Pub. No.: US 2019/0015098 A1
(43) Pub. Date: Jan. 17, 2019

(54) SUTURE LOCKING FREE NEEDLE

(71) Applicant: Mark Schamblin, Bakersfield, CA (US)

(72) Inventor: Mark Schamblin, Bakersfield, CA (US)

(21) Appl. No.: 15/646,548

(22) Filed: Jul. 11, 2017

(51) Int. Cl. A61B 17/06; A61B 17/04

(52) U.S. Cl. CPC A61B 17/06066; A61B 2017/0608; A61B 17/0483

(57) ABSTRACT

A suture locking free needle formed with an eye with a locking slot open to the eye. The locking slot secures a suture passed through the eye in order to prevent unthreading of the suture from the needle during use. The locking slot is narrower in width than the width of the corresponding suture so that suture forms an interference fit with the locking slot thereby preventing the suture from being released from the needle when the suture encounters the friction from tissue during use. In use, once the surgical suture is threaded into the surgical needle, the suture is pulled upward into the locking slot in order to hold it securely in place.

### FIELD OF THE INVENTION

The present invention pertains generally to a free needle for suturing tissue. More specifically, the present invention pertains to a suture locking free needle with an eye that locks the suture thread into place. The present invention is particularly, but not exclusively, useful as a free needle for suturing tissue or passing sutures in an operating room.

### BACKGROUND

Free needles are used in suturing tissue or passing sutures in the operating room. They are required when a free suture—a suture without a needle attached—is utilized in an operation. For example, implants often have a suture attached, which requires a free needle to then pass the suture through the adjacent tissue. Oftentimes a suture has been placed early in the surgical procedure, and the needle has been removed to protect the surgical team; later the suture will be re-introduced in the procedure and thus require a free needle.

Current free needle designs utilize an oval or circular shaped eye. The oval or circular eye has been the mainstay of needles since their development. This design, however, is inefficient in that it allows for the suture to fall, or pull, through the eye of the needle. As the needle is passed through the tissue, the friction of the tissue on the suture causes it to fall, or pull, through the eye, requiring the suture to be re-inserted into the eye of the needle with each pass through the tissue. This is time consuming, increasing the operative time, and leading to higher rates of infection, higher anesthesia complications, and increased risk to the patient. The necessity of reloading the needle numerous times also places the surgeon and surgical team at a higher risk of injuring themselves with the increased handling of a needle that has been involved in the procedure.

In order to minimize the risk of pull-through, a long thread is required to be pushed through the eye such that two thicknesses of the suture are pulled through the tissue, resulting in greater trauma to the tissue and requiring a much longer suture be used.

Another form of eye, called the French Eye, has been used with surgical needles. The French Eye provides a slit allowing for easy threading. The suture is pulled from outside the needle through the slit and into the eye. Ridges then hold the suture in place. Nonetheless, the risk of the suture unthreading is still present in the French Eye, which presents the additional disadvantage that it is appropriate only for finer suture threads and specific materials. A larger suture, for example, is too thick to pass through the slit.

In order to reduce the risk of unthreading, swaged ends are commonly used in suturing needles. Swaged needles are single-use needles with the suture permanently attached to the end of the needle. There are, however, many instances in which a free needle is necessary during an operation, and a swaged needle is unsuitable on those occasions.

In light of the above, it would be advantageous to provide a free needle with a mechanism for locking a suture thread in place while the suture is being passed through tissue. It would be further advantageous to provide a needle with a mechanism for locking a suture that could be specially sized to accommodate and lock a specific suture size from the range of 0.01 mm to 0.7 mm. It would be further advantageous to provide a free needle with a mechanism for locking sutures that could be configured to lock sutures of multiple sizes.

### SUMMARY OF THE INVENTION

The present invention is directed to a suture locking free needle that prevents the surgical suture, also referred to as surgical thread, from pulling through the eye of the needle during use. The suture locking free needle of the present invention has a body with a point end opposite an eye end. The point end of the free needle tapers from the main diameter of the body to form a point. In contrast, the eye end of the free needle expands from the main diameter of the body to enable the placement of an eye opening in the eye end.

Although the eye can be any shape which allows a suture to be passed through, it is generally a substantially circular or oval shape. As used herein, the width of the eye is measured transverse to the longitudinal axis of the suture locking free needle at the broadest opening of the eye. Thus, the width of the eye is equivalent to the diameter of the eye if the eye is formed as a circle.

A locking slot is formed into the eye end adjacent to and open at the eye. Some embodiments of the present invention have multiple locking slots, each locking slot formed with a distinct width. In all embodiments, the locking slot is narrower than the eye. The narrower width of the locking slot enables a suture passed through the eye of the needle to be secured in the locking slot.

The present invention embraces having an array of locking slots connected in a series, each having a locking slot width sized to receive and secure a suture of a particular size. The width of each locking slot is sized slightly smaller than the width of the suture intended for use in the operative procedure. For example, a locking slot sized to receive a 0.01 mm suture would be slightly smaller in width than 0.01 mm to enable and facilitate an interference fit between the locking slot and the 0.01 mm diameter suture. Each locking needle is labeled on needle packaging to inform the surgeon or surgeon's assistant of the appropriate size suture to use with the particular locking needle stored within the needle packaging. Locking slots can be properly sized to securely receive sutures ranging in diameter size from 0.01 mm to 0.7 mm.

In threading the needle, the suture is quickly passed through the eye, and then pulled (usually upward) into the locking slot. As the locking needle is passed through tissue, the frictional forces generated by the tissue on the suture are insufficient to overcome the frictional forces created by the locking slot on the suture. As a result, the suture stays secured within the locking slot thereby enabling the surgeon to focus on the surgical procedure with confidence that the suture will not pull free from the locking needle.

In an exemplary embodiment, the locking slot is placed along the longitudinal axis of the locking needle and extending from the eye in a direction opposite the body of the locking needle. Additionally, in an exemplary embodiment, the locking slot is U-shaped, meaning it has straight sides to form the locking pin width and terminating in a closed rounded end. Alternatively, the locking slot can taper to a point.

Alternative embodiments of the locking needle include multiple locking slots connected directly to the eye. In some embodiments, two locking slots are each connected to the eye and positioned opposite one another, with a first locking slot terminating away from the body of the needle and the second locking slot terminating toward the body of the needle. The locking slots can be U-shaped, tapered, or one of each. The locking slots may be the same size, or may be of different sizes in order to accommodate different-sized sutures.

In other embodiments, a least two locking slots are connected to and open at the eye and each of the locking slots are positioned so as to form an angle with respect to the longitudinal axis of the needle. The angle with respect to the longitudinal axis can range from 0 to 180 degrees.

Another alternative embodiment includes multiple locking slots connected in series to one other with each locking slot having a different width. The widest locking slot is connected directly to the eye, the second widest to the widest, and so forth. Each locking slot may have a tapered end connected to the next widest locking slot, except for the final locking slot, which may either taper to a point or be U-shaped.

### BRIEF DESCRIPTION OF THE DRAWINGS

The nature, objects, and advantages of the present invention will become more apparent to those skilled in the art after considering the following detailed description in connection with the accompanying drawings, in which like reference numerals designate like parts throughout, and wherein:

FIG. 1 is prior art image of a surgical needle;

FIG. 2 is a prior art image of a surgical needle having a closed eye;

FIG. 3A is a prior art image of a surgical needle having a French Eye;

FIG. 3B is a prior art image demonstrating the threading of a surgical needle having a French Eye;

FIG. 3C is a prior art image of a surgical needle having a threaded French Eye;

FIG. 4 is a prior art image of a surgical needle having a swaged end;

FIG. 5 is a perspective view of a surgical needle of the present invention, showing the needle point, body, and having an eye formed with a locking slot;

FIG. 6 is a front view of another surgical needle of the present invention showing a different curvature from that of FIG. 5;

FIG. 7A is a front view of the eye end of the suture locking free needle shown in FIG. 5 and showing the eye formed with a U-shaped locking slot;

FIG. 7B is a front view of the eye end of the suture locking free needle shown in FIG. 6 and showing, showing the eye formed with an alternative embodiment tapered locking slot;

FIG. 7C is a front view of the eye end of an alternative embodiment multi slot suture locking free needle, showing the eye formed with two tapered locking slots;

FIG. 7D is a front view of the eye end of an alternative embodiment multi slot suture locking free needle, showing the eye formed with a tapered locking slot and a U-shaped locking slot;

FIG. 7E is a front view of the eye end of an alternative embodiment multi slot suture locking free needle, showing the eye formed with two U-shaped locking slots;

FIG. 7F is a front view of the eye end of a suture locking free needle with an alternative embodiment locking slot and showing the eye formed with locking slot with a narrow neck and circular body;

FIG. 8 is a front view of the eye end of an alternative embodiment multi slot suture locking free needle, showing the eye formed three locking slots connected to each other in series with different slot widths;

FIG. 9 is a front view of the eye end of the suture locking free needle shown in FIG. 5 and showing a cross sectional view of a suture placed through the eye during threading and then positioned in an upward direction into the locking slot where the suture is slightly deformed in an interference fit with the locking slot and secured within the locking slot by frictional forces;

FIG. 10A is an isometric view of the suture locking free needle shown in FIG. 5 with a suture to be threaded;

FIG. 10B is an isometric view of the suture locking free needle shown in FIG. 10A threaded with the suture through the eye; and

FIG. 10C is an isometric view of the suture locking free needle shown in FIG. 10B with the suture pulled into the locking slot and locked into place.

### DETAILED DESCRIPTION

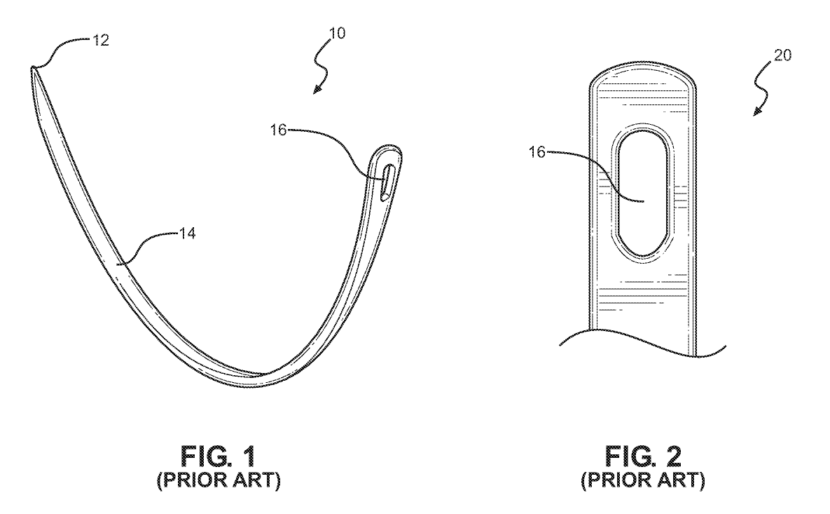

Referring initially to FIG. 1, prior art image of a free needle 10 is shown. Free needle 10 represents a standard surgical needle without a permanently attached suture. Free needles have a point 12 at one end, a shaft or body 14, and an eye 16 opposite the point 12. Surgical needles, including free needles, come in a variety of shapes, sizes, and curvatures. In some cases, a straight needle, or one that has no curvature, is used. A needle may have a round cross-section that tapers to a point, flat sides as in a “spatula needle,” a triangular cross-section, or other shape as appropriate to its particular intended use. The curvature of the needle generally ranges from ¼ circle through ⅝ circle, with J-shaped, compound curves, and straight needles are also used in some circumstances.

Referring now to FIG. 2, an enlarged image of a prior art closed eye is shown and generally designated 20. Closed eye needles generally include an eye 16 that has a circular or oval shape. In use, the suture is passed through the eye in order to thread the needle. In practice, the needle occasionally becomes unthreaded during use as the friction of the suture passing through the tissue pulls the suture from the eye. Despite these shortcomings, free needles are still necessary when a free suture is used in an operation.

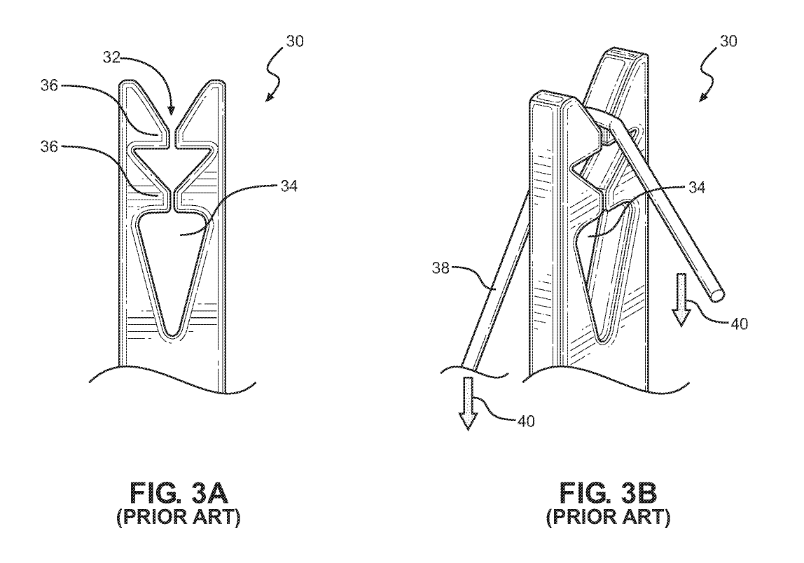

Referring now to FIGS. 3A through 3C, a prior art image of a surgical needle formed with a French Eye 30 and its use is shown. A French Eye 30 has a slit 32 from the end of the needle to the eye 34. Ridges 36 prevent a suture from falling back out of the needle through the slit 32 once the needle is threaded. To thread the French eye 30, a suture 38 is placed over the end of the needle and pulled in a downward direction 40. The suture passes through the slit 32 past the ridges 36 into the eye 34 of the needle. Once in place, ridges 36 prevent the suture 38 from passing back out of the needle through the slit 32. Although the suture 38 is held in place by the ridges 36 in the sense that it cannot pass back through the slit 32, the French Eye 30 still has the problem of unthreading through the eye 34 itself during an operation.

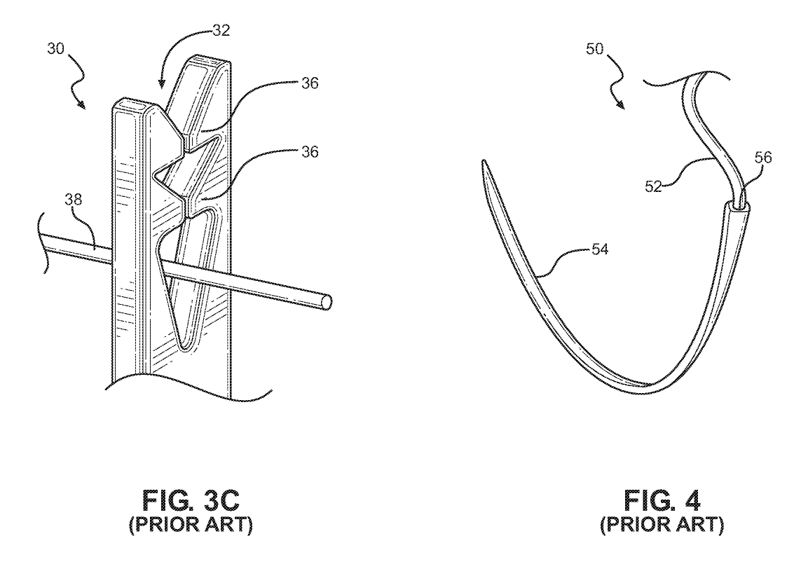

Referring now to FIG. 4, a prior art image of a swaged end of a swaged surgical needle is shown and generally designated 50. As opposed to a free needle, a swaged needle has a suture 52 permanently attached to the needle 54 via a swage 56. The swage 56 prevents the needle 54 from becoming unthreaded, but results in the needle 54 being appropriate only for a single use. Moreover, a swaged surgical needle 50 cannot be used with a free suture. Thus, the swaged surgical needle 50 failed to solve the problem of unthreading in those operations in which free sutures are required.

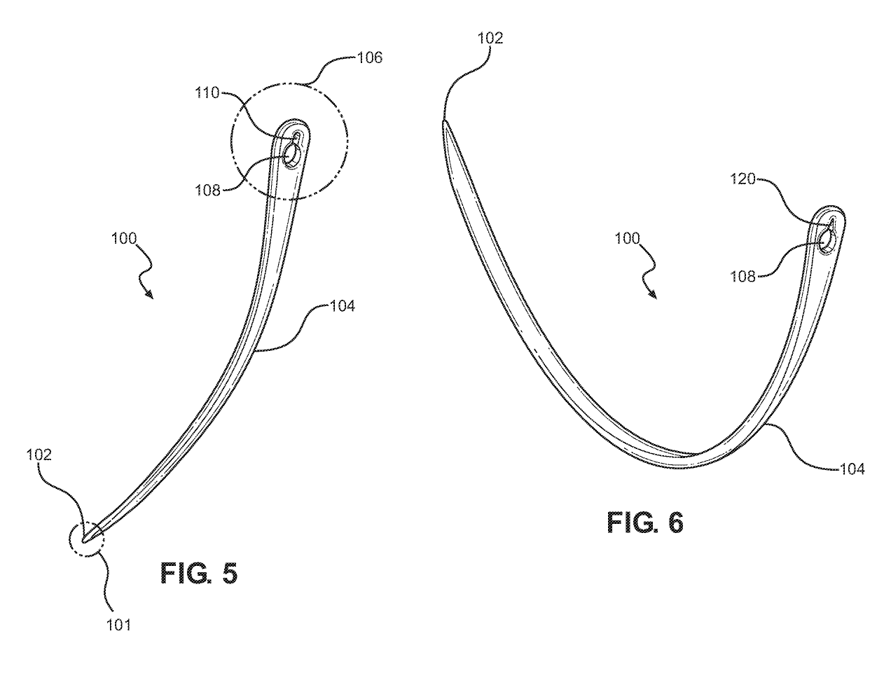

Referring now to FIG. 5, a suture locking free needle 100 of the present invention is shown. The suture locking free needle 100 has a body 104 having a point end 101 terminating a point 102 and an eye end 106 opposite the point end 101. The body 104 shown in FIG. 5 has a ¼ circle curvature. However, the body 104 can be straight or curved, and the invention described herein can be applied to any conventional needle shape or needle cross-section known in the art.

An eye 108 is formed in the eye end 106 of the body 104. A locking slot 110 is formed adjacent to and open at the eye 108. The locking slot 110 is narrower than the eye 108. The locking slot 110 is sized to receive and secure a specific size diameter suture 250 (shown in FIG. 9). Once the suture locking free needle 100 is threaded with a suture 250 (as shown in FIG. 9), the suture 250 is pulled into the locking slot 110, which holds the suture 250 in the suture locking free needle 100 while the suture locking free needle 100 is passed through tissue during a surgical procedure.

Referring now to FIG. 6, a suture locking free needle 100 of the present invention is shown with an alternative embodiment tapered locking slot 120. The tapered locking slot 120 facilitates the use of a single suture locking free needle 100 for sutures 250 ranging in size from 0.01 mm to 0.7 mm in diameter.

The suture locking free needle 100 shown in FIG. 6 is a ⅜ circle, however, as with the locking slot 110 of FIG. 5, the tapered locking slot 120 of FIG. 6 is adaptable to needles of different curvatures. More generally, embodiments of the present invention comprise straight needles and needles of all curvatures, shapes, and sizes, and each is fully contemplated herein.

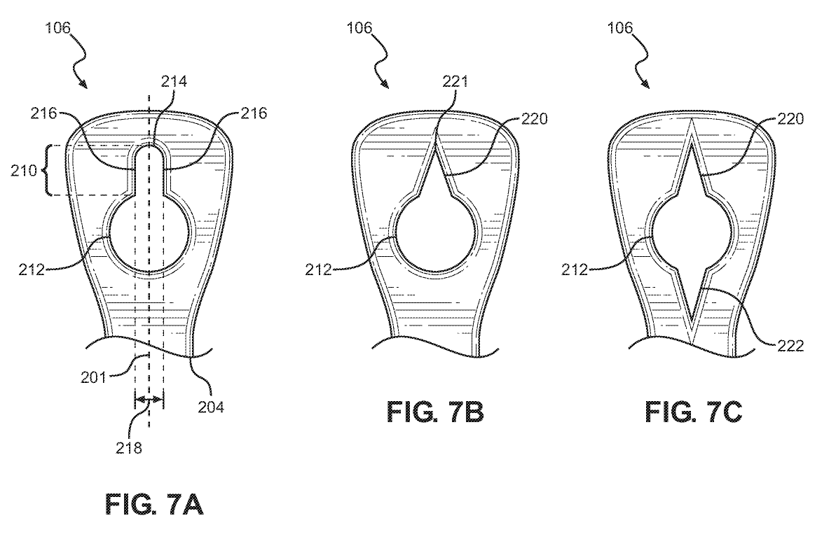

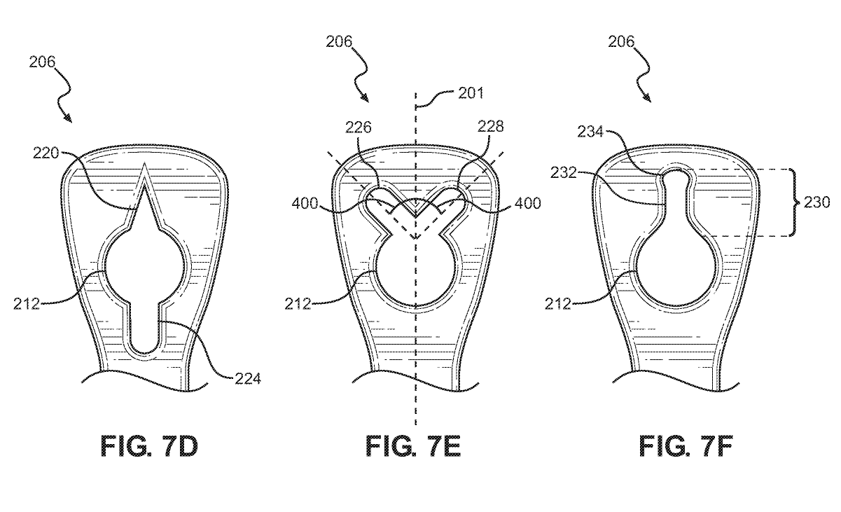

FIG. 7A shows the eye end 106 of a suture locking free needle 100 of the present invention with a U-shaped locking slot 210 on a substantially circular eye 212. The U-shaped locking slot has a curved end 214, comprising a curvature on its closed end, and parallel straight sides 216 extending from the curved end 214 to the base of the locking slot 210 where the locking slot 210 is joined to the eye 212.

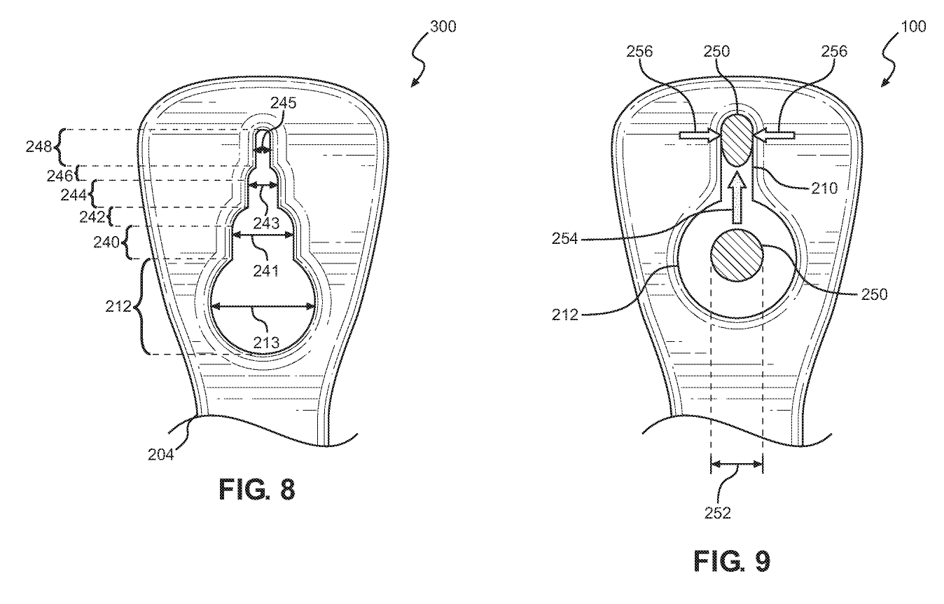

The width 218 of a locking slot of any shape is understood to be the width at its base, where the locking slot either connects either to the eye 212 of the needle, or to another locking slot as will be more fully described below in conjunction with FIG. 8.

FIG. 7B shows the eye end 106 of a suture locking free needle 100 of the present invention with a tapered locking slot 220 adjoining the eye 212 of the needle. The tapered locking slot 220 tapers from its width at the connection to the eye 212 to a point 221. The tapered locking slot 220 allows the needle to be used with a range of suture sizes.

FIG. 7C shows the eye end 206 of a multi-slot suture locking free needle 200 of the present invention with a first tapered locking slot 220 adjoining the eye 212, and a second tapered locking slot 222 also adjoining the eye 212. The first tapered locking slot 220 and the second tapered locking slot 222 are positioned opposite one another. The tapered locking slots 220 and 222 may be sized differently from one another or sized the same. When the tapered locking slots 220 and 222 are sized differently from one another, the locking needle is capable of being used with two ranges of suture sizes. In such cases, one locking slot is used for sutures of larger sizes, and the other is used for sutures of smaller sizes.

As seen in FIG. 7D, tapered locking slot 220 and U-shaped locking slot 224 are not the same shape. The tapered locking slot 220 is placed along the longitudinal axis of the multi-slot suture locking free needle 200 and extends from the eye 212 in a direction opposite the body 204 of the multi-slot suture locking free needle 200. The U-shaped locking slot 224 is placed adjacent to the eye 212 and along the longitudinal axis of the multi-slot suture locking free needle 200 and extends from the eye 212 in a direction toward the body 204 of the locking needle 200. The locations of the two locking slots may be reversed, or other shapes may be substituted for either or both locking slots.

As seen in FIG. 7E, where two locking slots are adjacent to the eye 212, it is not necessary for two locking slots to be located opposite one another. In FIG. 7E, a U-shaped locking slot 226 and a second U-shaped locking slot 228 are each located adjacent to and open at the eye 212 and each form an angle 400 with respect to the longitudinal axis 201 of the multi-slot suture locking free needle 200. The U-shaped locking slots 226 and 228 may be of the same size, or may be different sizes to allow the needle to accommodate different sizes of suture.

FIG. 7F shows the eye end 106 of a needle of the present invention with a locking slot 230 having a narrowing neck 232 connecting a circular body 234 to the eye 212. The neck 232 narrows from the width of the locking slot 230 at its base, and then extends outward into a circular body 234. The circular body 234 has a diameter greater than the narrowest portion of the neck 232, but less than or equal to the width of the locking slot 230 at its base.

Referring now to FIG. 8, the eye end 206 of a multi-slot reducing free needle 300 embodiment of the present invention is shown, with multiple locking slots 240, 244, and 248 connected in a series to the eye 212. As seen in FIG. 8, a first locking slot 240 is located adjacent to and open at eye 212. The first locking slot 240 has a first slot width 241, extends away from the body 204 of the needle 200 and terminates at a tapered end 242 opposite the eye 212. The tapered end 242 tapers from the first slot width 241 to a second slot width 243 of a second locking slot 244.

The second locking slot 244 is located adjacent to and open at the tapered end 242 of the first locking slot 240. The second locking slot 244 has a tapered end 246 opposite the eye 212 and first locking slot 240, which tapers down to a third slot width 245 a third locking slot 248. The third locking slot 248 is located adjacent to and open at the tapered end 246 of the second locking slot 244. Such a configuration enables a single multi slot needle 200 to be used with a variety of sizes of sutures 250. Although an exemplary embodiment includes three locking slots, it is also contemplated that two locking slots may be used in the fashion shown in FIG. 8, or four or more locking slots may be used, as appropriate to the circumstances. It is also contemplated that the locking slots may extend toward the body 204 from the eye 212 rather than away from the body 204.

The multiple locking slots, each having a different width, allow the needle to be used effectively with different suture sizes. When the suture is pulled from the eye 212 into the locking slots, it will remain held in place by the particular locking slot appropriate to its size.

Referring now to FIG. 9, a diagram of a surgical needle of the present invention is shown, depicting a suture 250 being locked into place. The suture 250 is passed through the eye 212 in the normal process of threading a needle. The diameter 252 of the suture 250 is slightly larger than the width 256 of the locking slot 210. The suture 250 is pulled in the direction 254 of the locking slot 210, until it passes from the eye 212 through to the locking slot 210. Due to the diameter 252 of the suture 250 being greater than the width 256 of the locking slot 210, friction on the suture 250 holds the suture 250 in place and prevents the suture 250 from unthreading from the needle.

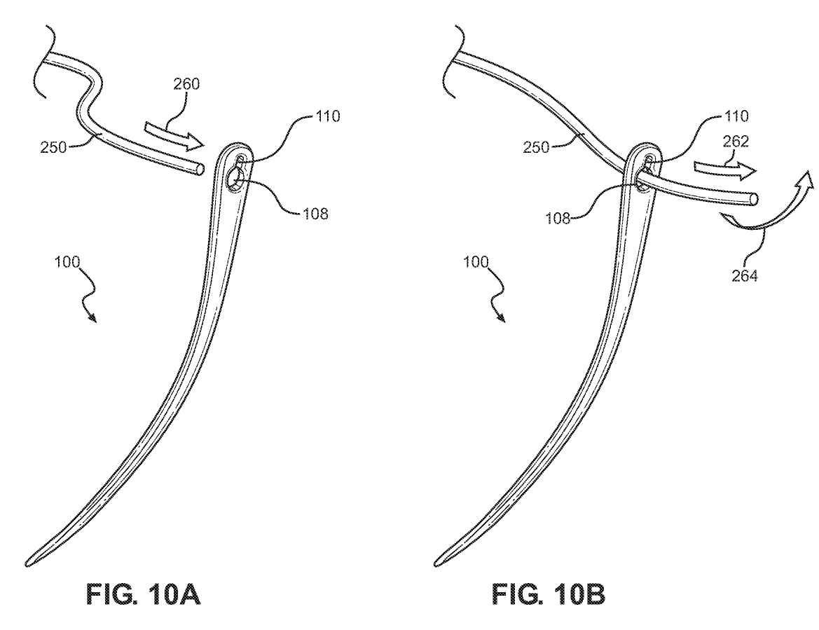

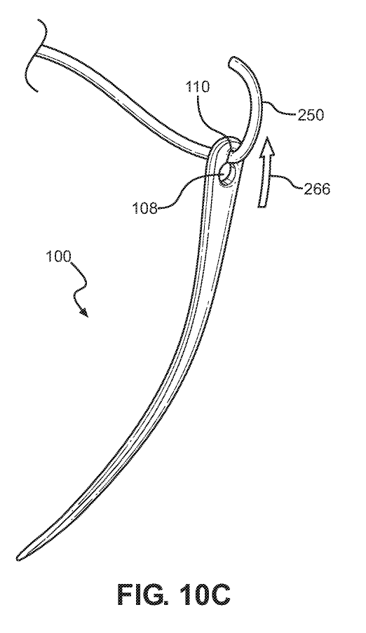

Referring now to FIGS. 10A through 10C, a method of use of the surgical needle of the present invention is depicted. Specifically, in FIG. 10A a surgical needle 100 of the present invention is shown ready for threading suture 250 through eye 108. Suture 250 is moved in direction 260 into the eye 108 of the needle 100. As shown in FIG. 10B, the suture 250 is then passed through the eye 108 of the needle 100 in direction 262, and then upwards in direction 264, such that suture 250 is pulled into locking slot 110 of needle 100. As shown in FIG. 10C, the upward force in direction 266 brings the suture 250 into the locking slot 110 where it is held in place. The needle 100 can then be passed through tissue without becoming unthreaded. This also allows the treating physician to minimize the length of suture 250 that extends through the needle 100, thus eliminating the need to pull two lengths of suture through the tissue being sutured. If necessary, suture 250 may be easily removed from locking slot 110 by pulling suture 250 opposite direction 266, and pulling it from the eye 208 to remove the needle during the procedure. The may be easily re-threaded when needed.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

While there have been shown what are presently considered to be preferred embodiments of the present invention, it will be apparent to those skilled in the art that various changes and modifications can be made herein without departing from the scope and spirit of the invention. Accordingly, the invention is not to be limited as except by the appended claims.

What is claimed is:

1. A suture locking free needle comprising:
a body having a point end terminating in a point and an eye end opposite said point end, said eye end formed with an eye and a locking slot adjacent and open to said eye;
wherein said locking slot is configured to hold in place a surgical thread pulled from said eye into said locking slot.

2. The suture locking free needle as recited in claim 1, wherein said eye has a first width and said locking slot has a second width narrower than said first width.

3. The suture locking free needle as recited in claim 2, wherein said eye has a substantially circular shape.

4. The suture locking free needle as recited in claim 3, wherein said locking slot has a U-shape.

5. The suture locking free needle as recited in claim 3, wherein said locking slot tapers to a point.

6. The suture locking free needle as recited in claim 3, wherein said locking slot tapers to a rounded tip.

7. The suture locking free needle as recited in claim 3, wherein said locking slot has a neck that extends outward into a circular body.

8. The suture locking free needle as recited in claim 3, further comprising a second locking slot adjacent to said eye.

9. The suture locking free needle as recited in claim 8, wherein said locking slot and said second locking slot are positioned opposite one another.

10. The suture locking free needle as recited in claim 8, wherein said locking slot has a U-shape and said second locking slot tapers to a point.

11. The suture locking free needle as recited in claim 8, wherein said locking slot and said second locking slot each has a U-shape.

12. The suture locking free needle as recited in claim 8 wherein said locking slot and said second locking slot are positioned adjacent to one another so as to each form an angle with respect to a longitudinal axis of said suture locking free needle wherein said angle is less than 180 degrees and wherein said locking slot and said second locking slot each terminate toward said eye end and away from said point end of said body.

13. A suture locking free needle comprising:
a body having a point end terminating in a point opposite an eye end, said eye end formed with an eye and a plurality of locking slots open to said eye;
wherein each said locking slot is configured to hold in place a surgical thread pulled from said eye into any of said locking slots.

14. The suture locking free needle as recited in claim 13, wherein said eye has a substantially circular shape.

15. The suture locking free needle as recited in claim 14, wherein said eye has an eye width and wherein each of said plurality of locking slots is formed with a locking slot width narrower than each said eye width.

16. The suture locking free needle as recited in claim 15, wherein said plurality of locking slots comprises a first locking slot with a first locking slot width, a second locking slot with a second locking width, and a third locking slot with a third locking slot width wherein said first locking slot width, said second locking slot with and said third locking slot width are each smaller than said eye width and are also different widths from one another.

17. The suture locking free needle as recited in claim 16, wherein said first locking slot terminates at and is open to said second locking slot and said second locking slot terminates at and is open to said third locking slot.

18. The suture locking free needle as recited in claim 17, wherein said first locking slot, said second locking slot and said third locking slot each terminate toward said eye end, opposite said point end of said needle.

19. The suture locking free needle as recited in claim 15, wherein said first locking slot is configured to hold in place a suture having a first size, said second locking slot is configured to hold in place a suture having a second size smaller than said first size, and said third locking slot is configured to hold in place a suture having a third size smaller than said second size.

20. A method of threading a suture locking free needle comprising the steps of:
(1) providing suture locking free needle comprising:
a body having a point end terminating in a point and an eye end opposite said point end, said eye end formed with an eye and a locking slot adjacent and open to said eye;
wherein said locking slot is configured to hold in place a surgical thread pulled from said eye into said locking slot;
(2) passing a first end of a surgical suture through said eye of said suture locking free needle; and
(3) pulling said surgical suture into said locking slot such that said surgical suture is secured in said locking slot.
